(19) United States
(12) Patent Application Publication
(10) Pub. No.: US 2011/0008527 A1
(43) Pub. Date: Jan. 13, 2011

(54) Solid Medication Tracking

(75) Inventors: Ross Teggatz, McKinney, TX (US); Wayne Chen, McKinney, TX (US)

Correspondence Address: MICHAEL T. KONCZAL, PATENT ATTORNEY, P.O. BOX 863656, PLANO, TX, 75086, US

(73) Assignee: TRIUNE IP LLC, Richardson, TX (US)

(21) Appl. No.: 12/832,588

(22) Filed: Jul. 8, 2010

Related U.S. Application Data
(60) Provisional application No. 61/223,938, filed on Jul. 8, 2009.

(51) Int. Cl. A61K 9/44; B23K 26/00; B65D 83/04; G06F 19/00; G08B 21/00

(52) U.S. Cl. 427/2.23; 219/121.69; 219/121.68; 221/199; 235/375; 235/385; 340/540

(57) ABSTRACT

The disclosed invention provides tools and techniques for marking data on the surface of solid medications and tracking the medication in various ways using the data. According to preferred embodiments, an optical reader may be used to discern the features of data recorded on the surface of medication in a micromatrix pattern. The data read by the reader is then used in the performance of tracking and/or control tasks.

### PRIORITY ENTITLEMENT

This application is entitled to priority based on Provisional Patent Application Ser. No. 61/223,938 filed on Jul. 8, 2009. This application and the Provisional Patent Application have at least one common inventor. The present application is also related to, and has one or more inventors in common with, application Ser. No. 12/646,529 filed on Dec. 23, 2009, which is hereby incorporated herein for all purposes by this reference.

### TECHNICAL FIELD

The invention relates to solid medication marking. More particularly, the invention relates to the marking and tracking of solid dosages of medication with small identifying marks and more extensive data.

### BACKGROUND OF THE INVENTION

It is known to mark solid medication, such as pills, tablets, and capsules, with a pill imprint code. An imprint code consists of alphanumeric text printed, engraved, embossed (or debossed) onto the surface of the medication. Imprint codes identifying solid oral dosage medication are a regulatory requirement of the United States Food and Drug Administration. Some medications also have other identifying markings, such as logos, letters, marks, symbols, internal or external cut outs, or identifying shapes and colors. In order to positively identify an individual medication, the imprint code may be compared with a listing of codes recorded in a printed or electronic database. There are several problems and potential problems with this approach to identification. The lookup system is prone to problems such as improper identification of the imprint code by the user, misreading of the database, a lack of listings for a given description by the user, and multiple listings under the same descriptions, to name a few. Also, due to space constraints on the medication, the information is typically limited to the identity of the medication. Information such as expiration date, lot code, manufacturer, and the like are not often provided. Additionally, since the available visible information is limited to that conveyed by size, shape, color, and only a few alpha-numeric characters, medications are susceptible to counterfeiting. Advances in pharmaceutical research and changing demographics are leading to an increase in the use of solid dosage medications. One significant challenge this trend produces is the need for monitoring drug interactions. With many patients, especially those being treated by more than one doctor for various types of ailments, there is a higher degree of risk of information concerning medications not being effectively tracked, thus potentially placing patients at risk from harmful drug interactions.

Due to the foregoing and possibly additional problems, improved marking apparatus and methods for solid dosage medication would be a useful contribution to the arts.

### SUMMARY OF THE INVENTION

In carrying out the principles of the present invention, in accordance with preferred embodiments, the invention provides advances in the arts with useful and novel tools and techniques for marking useful data on the surface of solid medications. Embodiments described herein include micromatrix markings applied by altering the surface of the marked object, e.g., by applying ink or ablating with a laser. Marks are preferably read with a machine reader. The embodiments described are intended to be exemplary and not exclusive. Variations in the practice of the invention are possible and preferred embodiments are illustrated and described for the purposes of clarifying the invention. All possible variations within the scope of the invention cannot, and need not, be shown.

According to one aspect of the invention, in an example of a preferred embodiment, a method for marking solid medication with a micromark includes steps for ablating a portion of the surface of the medication with a laser. The micromatrix pattern thus formed is subsequently read using a machine reader.

According to another aspect of the invention described herein, in preferred embodiments features of the micromatrix pattern formed on the surface of medication has a cell size within the range of about 5 μm to about 30 μm.

According to another aspect of the invention, some preferred embodiments include the use of an optical reader adapted to discern the features of the micromatrix pattern.

According to yet another aspect of the invention, examples of preferred methods include steps for comparing one or more features discerned in the reading step to data stored in a database.

According to another aspect of the invention, methods for marking and reading medications may also steps for communicating one or more messages to a user or database.

The invention has advantages including but not limited to providing one or more of the following features, improved imprint image quality, increased data content, improved database content, improved database access, improved authentication, counterfeit detection and prevention, and inventory tracking. These and other advantages, features, and benefits of the invention can be understood by one of ordinary skill in the arts upon careful consideration of the detailed description of representative embodiments of the invention in connection with the accompanying drawings.

### BRIEF DESCRIPTION OF THE DRAWINGS

The present invention will be more clearly understood from consideration of the description and drawings in which:

FIG. 1 is a perspective view of an example of solid medication illustrating preferred embodiments using micromatrix patterns on the surfaces of the medication;

FIG. 2 is a top view of an example of a preferred embodiment of a micromatrix pattern on a scale of approximately 5 μm to 30 μm; and

FIG. 3 is a schematic view of examples of preferred embodiments of micromarking and reading system components and method steps;

FIG. 4 is another schematic view of examples of preferred embodiments of micromark reading systems and methods according to the invention; and

FIG. 5 is a perspective view of a an example of a preferred embodiment of a solid medication deployment unit using micromarks according to the invention.

References in the detailed description correspond to like references in the various drawings unless otherwise noted. Descriptive and directional terms used in the written description such as front, back, top, bottom, upper, side, et cetera; refer to the drawings themselves as laid out on the paper and not to physical limitations of the invention unless specifically noted. The drawings are not to scale, and some features of embodiments shown and discussed are simplified or amplified for illustrating principles and features as well as advantages of the invention.

### DESCRIPTION OF PREFERRED EMBODIMENTS

While the making and using of various exemplary embodiments of the invention are discussed herein, it should be appreciated that the marking and machine reading of micromatrix patterns on the surface(s) of medications exemplify inventive concepts which can be embodied in a wide variety of specific contexts. It should be understood that the invention may be practiced in various applications and embodiments without altering the principles of the invention. For purposes of clarity, detailed descriptions of functions, components, and systems familiar to those skilled in the applicable arts are not included. In general, the invention provides one or more, and preferably a combination of, advances in identification, authentication, and tracking for pharmaceuticals. The invention is described in the context of representative example embodiments. Although variations and alternatives for the details of the embodiments are possible, each has one or more advantages over the prior art.

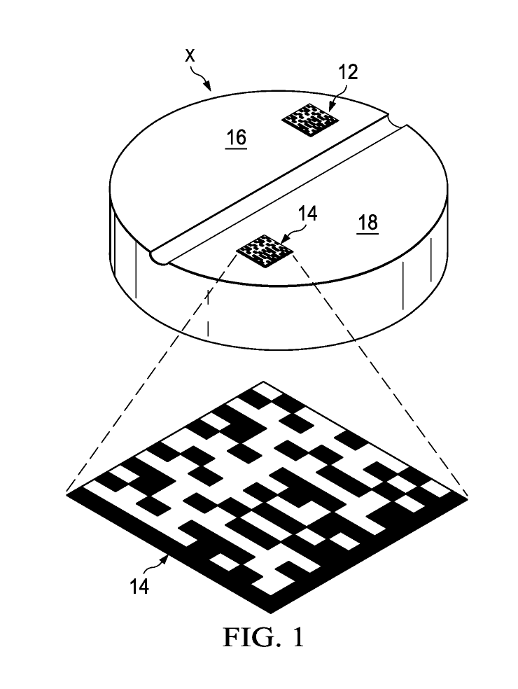

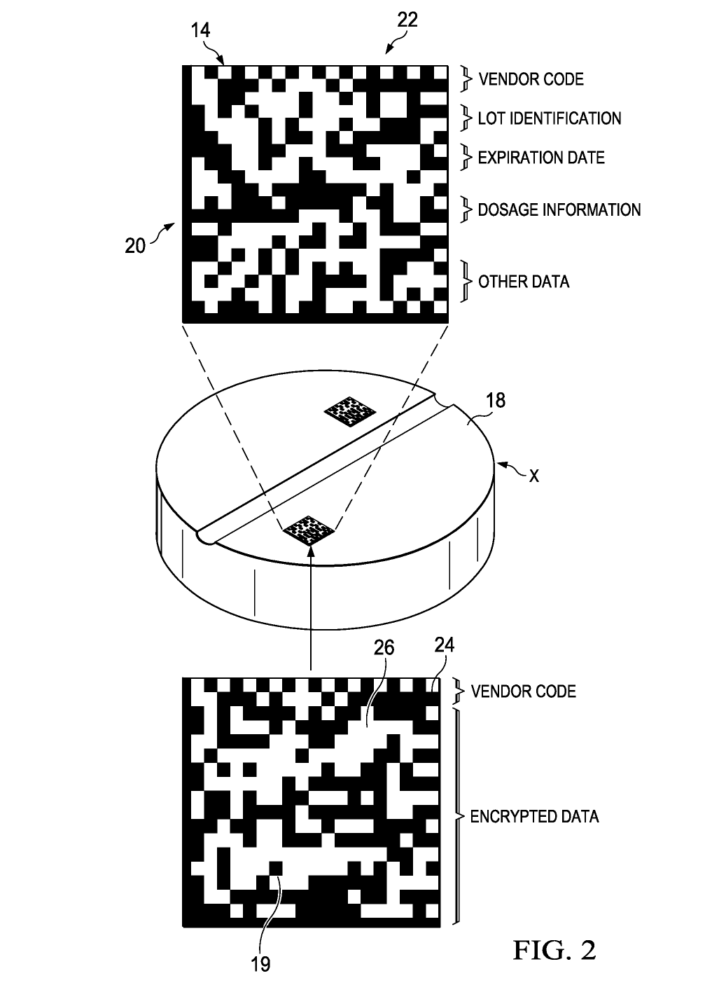

Referring initially to FIG. 1, a perspective view depicts an example of solid medication X (not part of the invention) exhibiting preferred embodiments of micromatrix patterns 12, 14 on the surfaces 16, 18 of the medication X. The preferred embodiment of micromarks 12, 14 shown provide a substantial amount of data, orders of magnitude beyond that conveyed by simple alphanumeric encoding. The micromarks used are preferably applied by a laser and capable of being read by a machine reader. The micromarks may alternatively be applied by other means for altering the surface of the item to be marked. For example, an optically readable ink may be applied. Preferably, during the manufacturing process, after the medication itself is substantially completed, a method such laser scribing or engraving is used to apply a small 2D micromatrix to one or more surfaces. The micromatrix preferably includes digitized data relating information such as, for example, medication name, manufacturer, expiration date, lot code, and security codes. The small size of the 2D micromatrix allows for this information to be imprinted on even the smallest doses of solid medication. Preferably, the micromatrixes are imprinted on multiple surfaces to facilitate reading the medication in various positions and also to provide redundancy. It is believed that the micromatrix markings would also act to deter counterfeiting due to the need for laser equipment and/or other technology to apply the micromarks. As shown in the close-up view of FIG. 2, the micromatrix pattern 14 preferably includes cells 19 arranged in rows 20 and columns 22 formed on the surface 18. The rows 20 and columns 22 each include selected regions of inscribed 24 and un-inscribed 26 surfaces. The inscribed and un-inscribed areas 24, 26 form a pattern of readable data. The data may include but is not limited to vendor codes, lot identification, expiration date, dosage information, and the like. The data may optionally be encrypted as well. In this example showing a preferred embodiment of a micromatrix 14 according to the principles of the invention, twenty rows 20 and twenty columns 22 are shown. Each cell 19 is about 15×15 μm, thus 400 bits of data are provided on a total area of approximately 900 square micrometers.

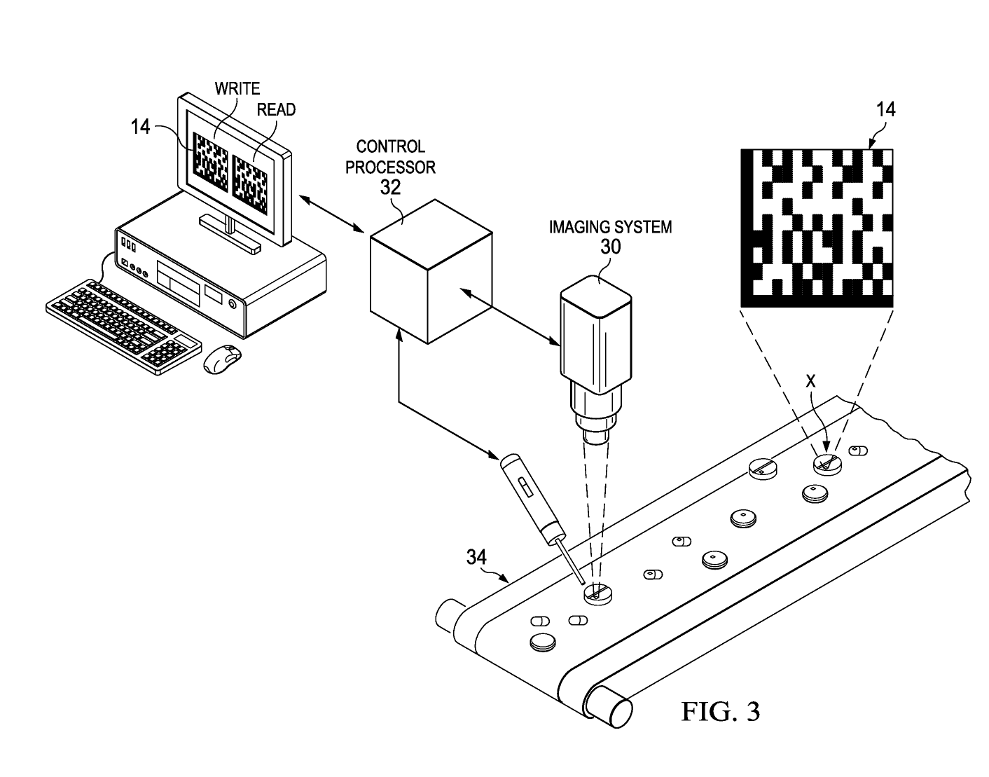

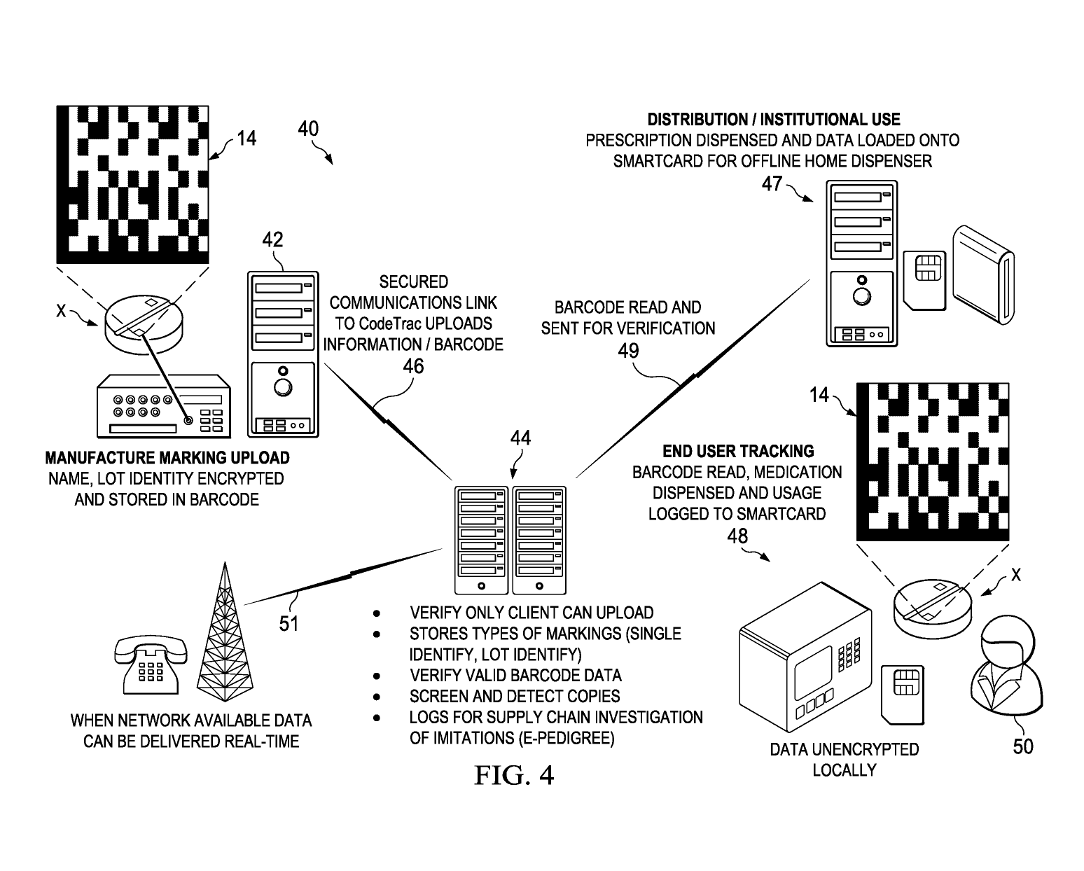

As further described elsewhere, a handler mechanically manipulates the medication for marking, preferably by a laser. As shown in the overview of FIG. 3, a reader 30 is configured to optically scan the micromarks 14 presented to it by a suitable handling system 34. Preferably, the reader 30 is equipped to optically discern the shape, size, and color of the medication as well as the data recorded in the micromark 14. A control processor 32 preferably includes a database linked to the reader 30 suitable for storing and manipulating data such as for example, data relating to the identity of general and specific types of medications, the potential interactions among medications, specific information relating to production lots, dates, and in some cases individual serial numbers for each dose. Additional information may include doctor and/or patient records, schedules, expiration dates, payment or insurance information, and the like. In preferred embodiments, the system also includes communications capabilities such that the stored database, the data read from the reader at any given time, e.g., individual pill data, and other databases, such as one residing at a medical facility, may be linked for the exchange of information. Another overview of the systems and methods of the preferred embodiments of the invention is depicted in FIG. 4. As shown in this conceptual overview, the system 40 includes micromarked 14 medications X. Preferably, the micromarks 14 are applied at the manufacturer, and uploaded to a local database 42 for further tracking. In this representative embodiment, the local database 42 is shown linked to a central database 44 through a secure communication link 46. A distribution database 47 may also be used to link 49 at least a portion of the central database 44 to a system including a reader, as shown at reference numeral 48. The communication link 49 may provide feedback to indicate inventory, sales, or other information. One-way links, e.g., 51, may also be provided, delivering updated information downstream to distributors, pharmacies, medical professionals, patients or others. Data such as prescriptions, dosages, etc. may also be transferred using digital media 53 such as smart cards and memory sticks. Readers 48 may be used at the final distribution point, e.g., a pharmacy, and/or at the end user 50.

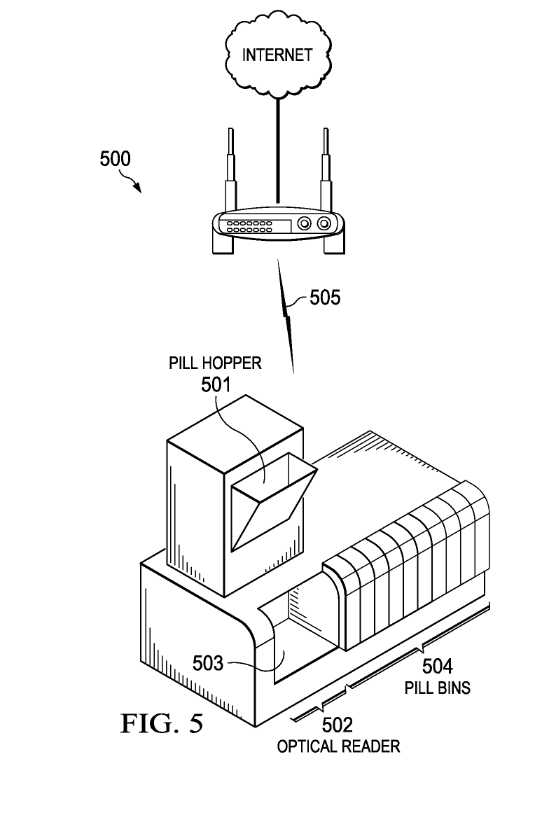

Additional aspects of the invention are illustrated in the preferred embodiment shown in FIG. 5. In another example of a solid medication deployment system according to a preferred embodiment of the invention, shown in the simplified view of FIG. 5, a solid medication deployment unit 500 has a medication hopper 501 for receiving a number of doses of various medications. A reader 502 is positioned at a staging area 503 where it receives medication from the hopper 501, reads the data marked on its surface, and passes it to one of various an individual containers 504. The individual containers 504 may be opened using a timer for dosage control, or may be directly accessible to users. Removable containers may also be used. The solid medication deployment system is scalable and may be used at the individual patient level to manage the administration of numerous medications, or may be implemented at the pharmacy, clinic, or other distribution level. Preferably, a local database (not shown) is provided, supplemented by a communication link 505, which may provide accessibility to virtually any communication device desired for a particular implementation. Preferably, the local database and communications functions are used for controlling the reading and dispensing and/or retaining of medications. For example, for tracking the need for refills, or for alerting caregivers to unexpectedly low inventories, indicating possible over dosage. The system may send an alert to an associated patient's cell phone or other device, reminding them to take their medication, seek a refill, or call their doctor. The medication database is preferably refreshed through the communication link and/or the use of portable memory devices provided by a pharmacy or other supplier. The system may also be linked to a patient or facility's payment information.

The micromatrix marking methods and systems of the invention provide one or more advantages including but not limited to one or more of, improved identification, authentication, tracking, coordination, and/or inventory control. While the invention has been described with reference to certain illustrative embodiments, those described herein are not intended to be construed in a limiting sense. For example, variations or combinations of features or materials in the embodiments shown and described may be used in particular cases without departure from the invention. Readers for example, may include handheld scanning units without departure from the principles of the invention. Although the presently preferred embodiments are described herein in terms of particular examples, modifications and combinations of the illustrative embodiments as well as other advantages and embodiments of the invention will be apparent to persons skilled in the arts upon reference to the drawings, description, and claims.

We claim:

1. A method for marking a dosage of solid medication with a micromark, comprising the steps of:
forming a micromatrix pattern on a surface of the medication by altering a portion of the surface; and subsequently,
reading the micromatrix pattern using a machine reader adapted for discerning the micromatrix pattern.

2. The method for marking a dosage of solid medication with a micromark according to claim 1, wherein the step of forming a micromatrix pattern on a surface of the medication further comprises ablating a portion of the surface with a laser.

3. The method for marking a dosage of solid medication with a micromark according to claim 1, wherein the step of forming a micromatrix pattern on a surface of the medication further comprises applying ink to a portion of the surface.

4. The method for marking a dosage of solid medication with a micromark according to claim 1, wherein the step of forming a micromatrix pattern on a surface of the medication further comprises forming features of the micromatrix pattern having a cell size within the range of about 5 μm to about 30 μm.

5. The method for marking a dosage of solid medication with a micromark according to claim 1, wherein the step of reading the micromatrix pattern further comprises using a reader adapted to optically discern the micromatrix pattern.

6. The method for marking a dosage of solid medication with a micromark according to claim 1, wherein the step of reading the micromatrix pattern further comprises using a reader adapted to optically discern the shape of the medication.

7. The method for marking a dosage of solid medication with a micromark according to claim 1, wherein the step of reading the micromatrix pattern further comprises using a reader adapted to optically discern the color of the medication.

8. The method for marking a dosage of solid medication with a micromark according to claim 1, further comprising the step of comparing one or more features discerned in the reading step to data stored in a database.

9. The method for marking a dosage of solid medication with a micromark according to claim 1, further comprising the steps of:
comparing one or more features discerned using the reader to data stored in a database; and
providing a message to a user.

10. The method for marking a dosage of solid medication with a micromark according to claim 1, wherein the reading step further comprises simultaneously reading a plurality of micromatrix patterns.

11. The method for marking a dosage of solid medication with a micromark according to claim 1, wherein the micromatrix pattern comprises medication content data.

12. The method for marking a dosage of solid medication with a micromark according to claim 1, wherein the micromatrix pattern comprises medication concentration data.

13. The method for marking a dosage of solid medication with a micromark according to claim 1, wherein the micromatrix pattern comprises medication source data.

14. The method for marking a dosage of solid medication with a micromark according to claim 1, wherein the micromatrix pattern comprises medication serialization data.

15. The method for marking a dosage of solid medication with a micromark according to claim 1, wherein the micromatrix pattern comprises medication compatibility data.

16. A micromatrix medication marking and reading system comprising:
marking apparatus for forming a micromatrix on the surface of medication by laser ablation of portions of the medication surface;
a reader for reading an encoded micromatrix formed by the marking apparatus; and
a handler for manipulating the medication.

17. A solid medication deployment system comprising:
at least one compartment for receiving medication in preparation for tracking;
an optical reader for reading encoded markings on the medication;
a database for tracking medication based on encoded markings read by the reader; and
means for dispensing medication subsequent to reading.

18. The system according to claim 17 further comprising communication means for communicating database information.

19. The system according to claim 17 further comprising communication means for communicating a message to a user.

20. The system according to claim 17 further comprising communication means for communicating control instructions to the means for dispensing medication.

21. The system according to claim 17 adapted for controlling daily medication dosages administered to a user.

22. The system according to claim 17 adapted for controlling medication inventory by a distributor.
